(19) United States
(12) Patent Application Publication
(10) Pub. No.: US 2003/0040795 A1
(43) Pub. Date: Feb. 27, 2003

(54) Anterior cruciate ligament reconstruction system

(76) Inventors: Robert J. Elson, Los Altos Hills, CA (US); Daniel Jacobs, Palo Alto, CA (US)

Correspondence Address: Johney U. Han, Morrison & Foerster LLP, 755 Page Mill Road, Palo Alto, CA, 94304-1018, US

(21) Appl. No.: 10/159,513

(22) Filed: May 31, 2002

Related U.S. Application Data
(60) Provisional application No. 60/295,389, filed on May 31, 2001.

(51) Int. Cl. A61F 2/08

(52) U.S. Cl. 623/13.12; 623/13.13; 623/902

(57) ABSTRACT

This discloses an orthopedic surgical device or system that may be used to reconstruct soft tissue, such as tendons and ligaments, within the knee or other parts of the body. More particularly, the device may be used to reconstruct the anterior cruciate ligament. Components of the device required for the reconstruction procedure are typically assembled as a unit outside the knee, and are introduced into a preformed bone tunnel as that unit. Components of the system and use of the system and its components are also described.

### CROSS-REFERENCE TO RELATED APPLICATIONS

This application claims the benefit of U.S. Provisional Patent Application Serial No. 60/295,389 filed May 31, 2001, which is incorporated herein by reference in its entirety.

### FIELD OF THE INVENTION

The present invention relates to the field of orthopedic surgery. Described here is an orthopedic surgical device or system that may be used to reconstruct soft tissue, such as tendons and ligaments, within the knee or other parts of the body. More particularly, the device may be used to reconstruct the anterior or posterior cruciate ligament of the knee. Components of the device required for the reconstruction procedure are typically assembled as a unit outside the knee, and are introduced into pre-formed bone tunnels as that unit. Components of the system and of using the system and its components are also described.

### BACKGROUND OF THE INVENTION

The anterior cruciate ligament (ACL) is a two-bundle ligament that helps to stabilize the knee joint. The ACL prevents posterior displacement of the femur on the tibia and hyperextension of the knee joint.

The ACL has poor healing properties, and thus, an untreated injury potentially leads to recurrent “giving-way” episodes, further damage to the menisci and articular cartilage, and possible progression to osteoarthritis (Brown et al., Clinics in Sports Medicine 18(1): 109-170 (1999)). Therefore, management of these injuries has evolved from nonoperative treatment through extracapsular augmentation and primary ligament repairs to the currently used open or arthroscopically assisted anterior cruciate ligament reconstruction. A complete understanding of the anatomy and biomechanics of the ACL has not been attained in the field of orthopedics, and thus, there is much active research in both normal and reconstructed knee biomechanics in order to develop improved systems for ACL reconstruction.

A typical surgical procedure for ligament replacement and reconstruction involves obtaining a tissue graft or a suitable synthetic graft to replace the damaged ligament. The graft may come from either another part of the patient's body (autograft), from a cadaver donor (allograft), or the graft may be synthetically manufactured. Current research may also lead to the use of grafts derived from animals (xenograft). In addition, the graft may itself be comprised entirely of soft ligament tissue or, alternatively, a combination of soft tissue attached to a “tendon bone block” on either end of the graft (a bone-tendon-bone graft). Methods for placement of such grafts are generally described in Goble et al., U.S. Pat. Nos. 4,772,286; 4,870,957; 4,927,421; 4,997,433; 5,129,902; 5,147,362; U.S. Pat. No. Re. 34,293; Kurland, U.S. Pat. No. 4,400,833; Jurgutis, U.S. Pat. No. 4,467,478; Hilal et al., U.S. Pat. No. 4,597,766; Seedhom et al., U.S. Pat. No. 4,668,233; Parr et al., U.S. Pat. No. 4,744,793; Van Kampen, U.S. Pat. No. 4,834,752; and Rosenberg, U.S. Pat. No. 5,139,520. Dore et al. teach the use of a tension spring for use as an artificial prosthetic ligament (U.S. Pat. No. 4,301,551).

Although the use of a bone-tendon-bone graft may provide the advantage of effective healing due to the efficient biointegration of the bone graft to the bone host, the harvesting of a bone-tendon-bone graft typically results in extensive morbidity to the donor knee joint, thus lengthening the patient's resumption of normal physical activity. It is, therefore, often preferable to harvest grafts made up entirely of soft tissue, e.g., a hamstring tendon, because such a procedure involves less donor site morbidity. On the other hand, it has historically been more difficult to effectuate and maintain accurate fixation of such grafts throughout the healing period where high-tension forces of the knee may act to disrupt the graft construct via, e.g., slippage from the fixation devices or via graft failure.

When performing ACL reconstruction with a soft tissue graft, the selected material is attached (fixated) to the natural insertion sites of the patient's damaged ligament. Many devices and procedures used for orthopedic ligament reconstruction are specifically designed both to overcome the myriad difficulties of fixating soft tissue ligament grafts to the hard tissue bone surface and to enable the patient to return to a full range of activity in as short a period of time as possible. To that end, medical researchers have attempted to duplicate the relative parameters of strength and flexibility found in natural ligaments of the body. Unfortunately, many existing procedures have proven inadequate for immediately restoring adequate strength and stability to the involved joint. Furthermore, even if immediate achievement of knee stability is achieved, many current methods are ineffective at maintaining such stability throughout the period when the mechanical phase of graft fixation is ultimately superceded by a permanent biological phase of graft integration.

One difficulty in effectively implanting a fully effective ligament reconstruction is the surgeon's need to balance a number of variables leading to “trade-offs”. Such variables include the need to position a sizable graft ligament at a precise location within the joint while minimizing trauma to the host bones and while constrained by the need to use the smallest possible bone tunnel. When creating the ligament reconstruction, it is generally important to use as large a graft ligament as possible, to (i) provide high graft strength along the length of the graft to prevent subsequent rupture, and (ii) provide an extensive supply of collagen material to facilitate effective integration of the graft ligament into the bone. At the same time, the physics of the knee joint dictate the location of the graft fixation points and hence the location of the bone tunnels. Of course, the particulars of the surrounding anatomy may affect graft ligament size and/or bone tunnel size.

Minimization of the size of the bone tunnel is important, since (i) larger bone tunnels are more destructive of the host bone, (ii) larger bone tunnels are more difficult to revise later in the event of graft failure, and (iii) desired osseous obliteration of the drilled tunnel by bony in-growth may be delayed, possibly leading to a weaker ultimate reconstruction. The particulars of the surrounding anatomy may also affect graft size and/or bone tunnel size.

The ability to determine the final resting tension on the ACL reconstruction system is thought to be important for determining ultimate joint stability after healing. Many systems allow determination of tension before and during insertion of the graft, but not subsequent to fixation and anchoring. Thus, the final intra-operative resting tension on the graft ligament is either unknown or unadjustable. Ideally, both ends of the graft ligament are anchored in tunnels situated and fixated respectively in the tibia and in the femur followed by adjustment of the ligament tension. In particular, the graft ligament should be tight enough to provide stability to the joint rather than being simply a “checkrein” incurring a load only at the extremes of motion. Anchor structures, such as those in Johnson (U.S. Pat. No. 5,562,668), are complex, bulky, and difficult to use properly. Methodologies for “pretensioning” the graft prior to fixation are shown in Daniel et al. ('542) and in Goble et al. (U.S. Pat. Nos. 5,037,426; 5,713,897).

Incremental adjustment of the tension on the graft after its insertion while maintaining the joint's range of motion and stability is a continuing challenge. As mentioned above, Johnson '668 shows a tensioning device for ligament grafts. One end of a patellar bone block graft is affixed to a carrier having a threaded cylindrical member extending toward the outer surface of the joint. The carrier is seated within a “thimble.” After the carrier is placed within the thimble, the surgeon adjusts the thimble to vary the tension on the graft. The Johnson '668 device appears to have inherent limitations. For example, once one end of the graft is anchored, any tension adjustment would necessarily twist the graft and therefore induce a torsional load on the graft.

Another variable to be addressed involves the balance between selecting an appropriate anchoring location to maintain position of the reconstruction devices on the one hand and on the other hand selecting a method and position of fixating the soft tissue so as to approximate it to the bony surface for healing. The available procedures may be separated into two general categories, those that permit anchoring of the device within the bone tunnel and those that utilize anchoring outside of the bone tunnel. External anchoring provides an advantage in that a substantial portion of the load on the graft may be borne by the strong bone exterior or cortex. However, such external anchoring presents several problems, including the requirement of a long graft to be harvested in order to reach the external fixation point; the presence of a long segment of stretchable graft within the bone tunnel that can widen the tunnel, impede healing, and damage the graft (bungee cord effect); and the lack of immobilization of the graft at the articular orifice that can lead to lateral motion (windshield or sundial effect), widening of the orifice, impeded healing, and damage to the graft.

Anchoring within the tunnel overcomes both of these obstacles but, in turn, diminishes the strength of the graft anchor. Internal anchoring typically requires the use of devices that are destructive of the soft graft tissue (as described below). Finally, anchoring the ligaments entirely within the bone tunnel precludes the surgeon from properly adjusting the tension on the graft after it has been placed within the tunnel.

Devices that are currently used for anchoring the graft include pins, screws, baffles, bone blocks, staples, and washers. The use of “cross-pinning”, i.e., in which a pin, screw, or rod is driven into the bone transversely to the bone tunnel intersecting and “cross-pinning” a bone-tendon-bone or soft tissue graft in the bone tunnel, to secure a graft is generally utilized for securing bone-tendon-bone grafts rather than soft tissue grafts.

As described above, a well-established method of maintaining a replacement graft at an anchor site entails the retention of the graft within the bone tunnel by an endosteal fixation device, such as an interference screw, to press at least one end of the graft against the interior wall of a bone space (Mahony, U.S. Pat. No. 5,062,843; Roger et al., U.S. Pat. No. 5,383,878; Steininger et al., U.S. Pat. No. 5,425,767; Huebner, U.S. Pat. No. 5,454,811; Laboureau, EU 0 317 406). Grafts may be anchored between two elements, the inner one being deformable (U.S. Pat. No. 5,108,431), and they may be passed through a center of a device, creating tension by relative movement of elements (DeSatnick, U.S. Pat. No. 5,571,184). However, such devices may create a gap between the bone and the ligament graft, thereby precluding maximal graft-tunnel contact at the point of immobilization, thus possibly impeding healing.

Interference screws, by definition, function by creating a tight fit between the graft and the surrounding bone. Such constructs require a continuous high-pressure load against both the graft and the surrounding bone, thus possibly leading to damage to the graft and erosion of the bone. Puncturing, piercing, and possible tearing of the graft is even more likely due to the additive loads present during flexion or extension of the knee or during high stress activities. This results in impeded healing or loosening of the interference fixation, and thus loss of fixation and graft slippage. Such an outcome could represent a failure of the operative procedure.

As mentioned above, other procedures allow a surgeon to anchor the graft outside of the bone tunnel and to the external bone surface. These procedures typically require the surgeon to use a graft having a length such that it extends beyond the cortex of the bone tunnel, bends at approximately a 90° angle so that the graft end is flush against the external bone surface for securing to the external bone. Stainless steel staples, buttons with sutures, and other related fixation devices have each been used for external anchoring.

### SUMMARY OF THE INVENTION

Described is a device or system (and its components) that reconstructs or replaces soft tissue ligaments found in a joint such as the knee. The system is installed in pre-formed bone tunnels made in the tibia and femur. One variation of the device may comprise a distal (typically, femoral) component or subassembly having, e.g., an anchoring member, and an opening for passage of a soft tissue graft. The anchoring member may be configured such that once it has been introduced into a bored hole within the bone, the member engages the femoral cortex to secure the device in the femur. The reconstruction system may also have a proximal (typically, tibial) component or subassembly having a bone anchoring portion and a graft-fixation portion. The bone anchoring portion may have a seat component. In this variation, pin plates may be positioned at the distal end of proximal graft fixation component for securing the ends of the soft tissue. The proximal end of the pin plates may slidably fit into the anchoring component, which anchors the pin plates within the tibia. The entire device may be placed in pre-formed bone tunnels, e.g., in the tibia and femur, as a single unit.

A tool may be used to adjust graft tension/length at the tibial end of the bone tunnel. A load cell may also be incorporated to indicate the amount of tension being applied. Once the appropriate graft tension has been set, the tibial assembly may be locked in place.

Another variation may use the same or a similar distal (again, often the femoral) subassembly as above. The proximal subassembly (usually tibial) may comprise a bolt, screw or other helical device, adjustably secured to a tibial seat with a fastener. This variation may also be constructed entirely ex vivo and then inserted within a patient as a completed unit.

In this variation, the terminal ends of graft may be secured between the shank of the bolt and a ring fastener, which is slidably adjustable along the shank of the bolt. The grafts may also be preconditioned prior to placement of the ring over the grafts. With the grafts positioned within the ring, the bolt may be pushed through the ring opening such that the grafts become wedged or pressed between the inner surface of the opening and the bolt. The bolt assembly, along with the grafts and ring may then be inverted such that the grafts are now outside the ring (i.e. the presentation surface), thus maximizing recipient bone to ligament graft contact. The bolt may then be positioned within the tibial seat and secured with a fastener. Alternatively, if ring is not inverted, the tibial subassembly may include one or more rings. In this embodiment the grafts are not external to rings, so the head of the bolt creates the presentation surface.

Pulling the bolt proximally through the tibial seat tightens the grafts. This may be accomplished by using an installation tool or by tightening the fastener against the tibial seat. Accordingly, the tension in the grafts may be adjusted post-deployment, i.e., after the device has already been positioned within the knee intra-operatively and potentially postoperatively to adjust the tension as needed.

### BRIEF DESCRIPTION OF THE DRAWINGS

FIG. 1 is a perspective view of a variation of a graft reconstruction device.

FIG. 2 is a perspective view of a variation of a soft tissue reconstruction device with a graft looped through the femoral assembly and the tibial assembly.

FIG. 3 is a cross-sectional view of a variation of a soft tissue reconstruction device within a bone tunnel.

FIG. 4 is a cross-sectional view of a variation of a soft tissue reconstruction device along with a sheath, spanner wrench, and load cell within a bone tunnel.

FIG. 5A is an isometric assembly view of another variation which may be used without a tooling rod.

FIG. 5B is an isometric view of an assembly for securing grafts.

FIG. 5C is an isometric view of the assembly from FIG. 5B prior to installation through a tibial seat.

FIG. 5D is an end view of grafts held between a ring and a bolt.

FIG. 5E is an isometric view of a tooling assembly which may be used to pre-stretch the grafts.

FIG. 5F is a cross-sectional view of a variation of the tibial assembly.

FIG. 6 is an exploded assembly view of the tibial assembly variation.

FIGS. 7A and 7B show side and cross-sectional side views, respectively, of a bolt which may be used to tension grafts.

FIG. 7C shows a side view of another bolt variation having an optional tapered section to help distribute stress within the grafts.

FIGS. 8A to 8C show isometric, top, and cross-sectional side views, respectively, of one variation of the ring.

FIG. 8D shows a cross-sectional side view of another ring variation having a tapered inner diameter.

FIGS. 9A to 9D show various views of one variation of the tibial seat.

FIG. 9E shows a cross-sectioned tibial scat positioned in a low profile within the bone and the forces acting upon the seat.

FIGS. 10A to 10D show various views of another variation of the tibial seat.

FIGS. 11A to 11C show isometric, top, and cross-sectional side views, respectively, of one variation of the fastener.

FIGS. 12A to 12C show isometric, top, and cross-sectional side views, respectively, of another variation of the fastener.

FIGS. 13A to 13C show side and cross-sectional views of one variation of a tool which may be used to adjust the tension in the grafts.

### DETAILED DESCRIPTION OF THE INVENTION

Described here is a device, often termed a “system,” used to reconstruct soft tissue in a pre-formed bone tunnel made in the knee. The device may be used for anterior or posterior cruciate graft (ACL or PCL) reconstruction. Generally, elements of the inventive device and the soft tissue are installed as a single unit within pre-formed tunnels of the tibia and femur. The elements of the device may be “bio-integrated” in that they are chosen and designed to cooperate in promoting optimal anchoring, fixation, and healing of the soft tissue graft. The system is further designed for ease of installation by a surgeon.

We mean the term “soft tissue” as used here to include any type of connective or muscular tissue, but in particular refers to tendons and ligaments. The soft tissue may be autologous, allogeneic, xenogeneic, artificially engineered, or include mixtures thereof, but generally includes tissue that is suitable for anterior cruciate ligament reconstruction. For instance, suitable ligament xenografts are described in U.S. Pat. No. 6,110,206 to Stone, and tissue-engineered tendons and ligaments are disclosed in U.S. Pat. No. 6,023,727 to Vacanti et al.

In this description, we have chosen to use terminology such as “distal” and “proximal” to refer to the positioning of the various portions of each variation of the system as they relate to their introduction into the bone tunnel or to their positioning relative to the surgeon implanting that system. Although it is likely that the “distal” position most often will be the end situated in the femur, and therefore the “proximal” end will be in the tibia, we wish to make clear that because of its design, the system may also be inserted from the femur to the tibia. In sum, “distal” sections may be femoral or tibial sections and “proximal” sections may also be femoral or tibial sections; “femoral” or “tibial “sections” may be either distal or proximal.

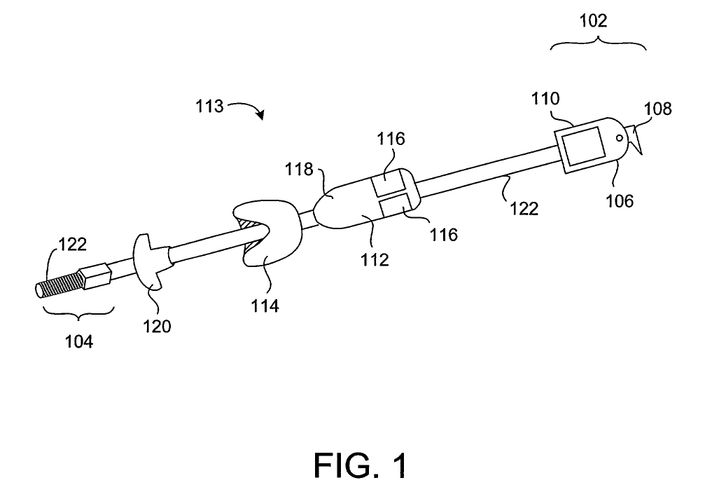

FIG. 1 shows one variation of the system used for soft tissue reconstruction. Reconstruction device or system (100) has a distal end (102) and a proximal end (104). The distal end (102) includes a femoral assembly (106) that has a pivotable member (108) at the femoral assembly distal end (102) and an opening for passage of a soft tissue graft (110) at the femoral assembly proximal end (104). Pivotable member (108) may be configured such that once the system has been introduced into a bored hole within the bone, member (108) engages the femoral cortex to secure the reconstruction device (100) in the femur. Pivotable member (108) is designed so that it may be introduced in a single direction axially through the bone tunnel, but upon “activation” of the member (108), it either becomes affixed to the bone or will not return through the tunnel unless “deactivated.” The concept of activation is quite simple, the member is remotely made to grasp or affix to the walls of the bone tunnel. For instance, the pivotable member (108) may be configured as a winged pawl or like structure. Alternatively, it may also be configured as ant actively expandable member having radially extending arms for grasping onto the walls of the bone. Generally, there is only one pivotable member that rests against the femoral tunnel wall, but more than one may be used if so desired, e.g., to engage opposing sides of the femoral cortical surface in the tunnel.

The reconstruction device (100) also includes a proximal (or tibial) assembly (113) that may comprise a soft tissue graft attachment component (112) and a seat component (114). Pin plates (116) are shown at the distal end of graft attachment component (112) for securing the ends of the soft tissue thereby forming a loop graft when pressed against the tissue. The proximal end (118) of graft attachment (112) slidably fits into the seat component (114) which anchors the graft attachment (112) within the tibia by the locking of a screw (120). Screws with various body lengths may be used depending on the amount of tension and/or length of graft required. The femoral assembly (106), graft attachment (112), seat component (114), and screw (120) are preferably placed in a preformed bone tunnel in the knee by tooling rod (122).

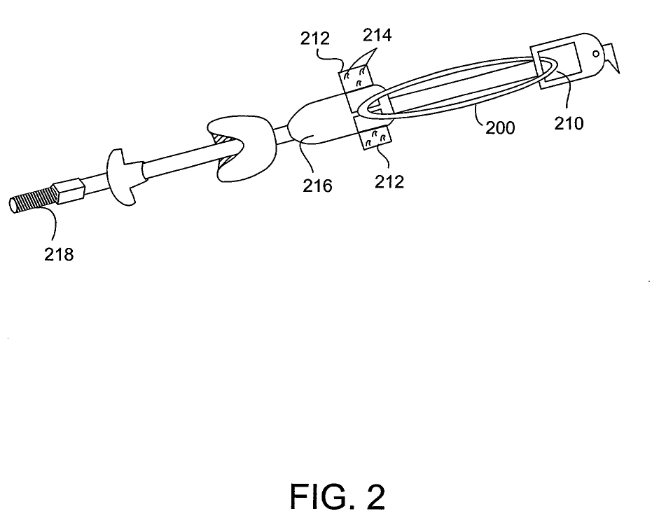

FIG. 2 shows the reconstruction device or system (100) with a loop graft of soft tissue (200) that has been looped through opening (210) of the femoral assembly. In this instance, a single graft is shown passing through opening (210) and returning upon itself to form a closed loop of graft extending between tibial assembly (113) and attachment component (112). Multiple loops and multiple grafts may also or instead be used. The soft tissue used to form loop grafts include any of those mentioned above. The loop of soft tissue may be made by passing a first end of the graft through an opening (210) at the femoral assembly proximal end, and then overlapping the first end with the second end of the soft tissue graft to form a loop. The overlapping ends are secured to one another to form a loop by closure of one or more pin plates (212). Alternatively, rather than looping the graft, individual grafts may be extended between the tibial assembly (113) and attachment component (112) and fixed at both ends to each respective assembly through various mechanical fixation methods, e.g., via piercing tines, crimped rings, sutures, etc. A plurality of attachment points (214) reside on the internal surface of pin plates (212), and upon pressing pin plates (212) onto the ends of soft tissue (200), the attachment points (214) may grasp the ends of the soft tissue to hold them together. Generally, these attachment points will be referred to a “tines”, “prongs”, or “barbs”. These tines will refer both to points that are sharp, i.e., able to separate tissue fibers, or blunt, i.e., not able to separate tissue.

The shape of the soft tissue points or barbs may be varied depending upon the size and type of tissue requiring grasping. Various tines or barbs may be canted or erect. Various shapes include those approximating arrowheads, hooks, nails, sharpened pencils, or the like. The attachment points may be canted to a selected direction or may be radial in direction. They may also be barbed, double barbed, etc. Further description relating to these barbs or tines may be found in U.S. patent application Ser. No. 09/574,603, filed May 19, 2000, the entirety of which is incorporated by reference.

The reconstruction device itself and its various components may be made of various biocompatible metallic components, e.g., stainless steel, titanium, nickel-titanium alloys, etc. The device and its individual components may alternatively be made of one or more compatible polymers. Biodegradable polymers synthesized from monomers comprising esters, anhydrides, orthoesters, and amides are suitable for use as biodegradable materials. Specific examples of biodegradable polymers include polyglycolide, polylactide, poly-α-caprolactone, polydioxanone, polyglyconate, copolymers of polylactide and polyglycolide, and the block and random copolymers of these polymers. Copolymers of glycolic, lactic, and other a-hydroxy acids may also be used. Porous materials and/or composites of absorbable polymers and ceramics, e.g., hydroxyapetite, are also suitable for use. Although the components of the device may comprise a single polymer or copolymer, generally for ease of construction by molding, the invention is not so limited. For example, the inventive device may be made of two or more types of polymers or copolymers, or of differing molecular weights of the same polymer or copolymer. For instance, the pin plate or ring materials might be produced from a more flexible polymer and the points, tines, or bolt from a stiffer material.

Generally, the reconstructive system so reconstructs soft tissue while minimizing the risks of graft failure. This device is typically installed as a single unit and thus provides a much simpler technique that often reduces the occurrence of surgical error.

The reconstruction device may be introduced into a pre-bored bone tunnel as a single unit. Such a tunnel may be constructed by use of various conventional surgical drills. The device may be introduced from the tibial end of the bone tunnel but is designed so that introduction from the femoral end of the bone tunnel is also possible. In one variation, prior to insertion into the knee, a graft such as a hamstring graft, is looped through an opening in the femoral assembly proximal end (210) as discussed above, and then pre-loaded with tension. While maintaining tension, the ends of the graft are looped through element (216) and secured by closure of the pin plate (212). Pretensioning of the graft helps to reestablish and maintain the normal stability of the joint by eliminating pivot shift and restoring articular movement to within normal values. During pre-insertion construction and tensioning of the reconstruction system, preparation of the knee for arthroscopic ACL reconstruction is completed, as described for example, in Jakob and Staubli. (1992). The Knee and the Cruciate Ligaments. Springer-Verlag, New York.

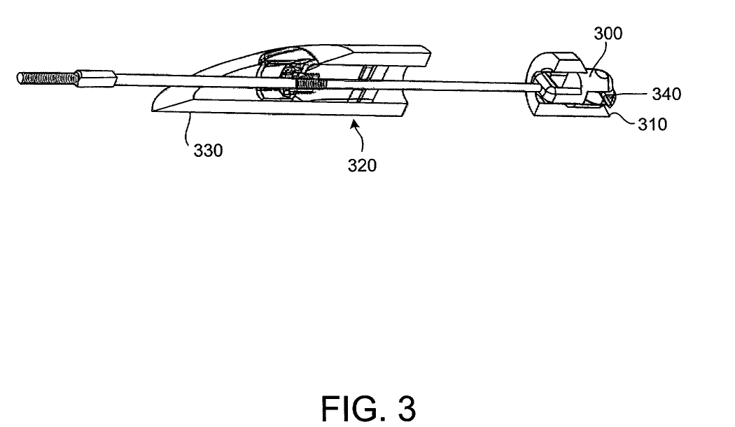

Once the graft has been properly conditioned, tooling rod (218) may be inserted into the device components and placed into the tunnel bored in the knee from the tibial cortex to the femoral cortex as a single unit. As illustrated in FIG. 3, the femoral assembly (300) (the graft is not shown for clarity) may be joined to femoral cortex (310) by insertion of the device (320) from the tibial side of the bone tunnel (330) beyond the femoral cortex (310) and then palpating the femoral assembly (300) to ensure it engages the femoral cortex (310) via rotation of winged pawl (340) when pulled back towards the tibia. Winged pawl (340) preferably rests against cortical bone surfaces with an amount of pressure preferable to maintain healing, i.e., an amount not sufficient to cause bone erosion, necrosis, and subsequent loss of fixation.

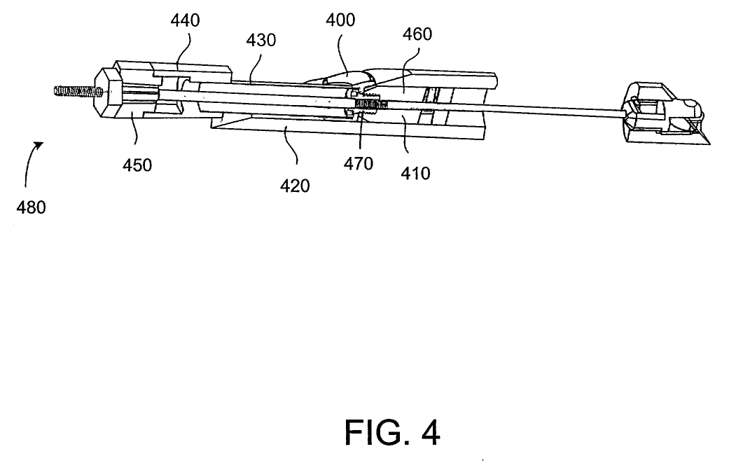

In FIG. 4, tibial seat component (400) preferably anchors graft attachment component (410) to the tibial cortex (420). A sleeve (430) with spanner wrench (440) on one end may be used to adjust graft tension/length at the tibial end of the bone tunnel. A load cell (450) may also be positioned adjacent to the base of spanner wrench (440) to indicate the amount of tension being applied by the spanner wrench (440). Once the appropriate graft tension has been fixed, tibial assembly (460) is locked in place by tightening of a screw (470) and tooling rod (480), along with spanner wrench (440) and load cell (450), may then be removed. The tooling rod (480) permits installation of this variation of the system by simply grasping the end of the prestretched assemblage and pressing it into the pre-bored bone tunnel, much as one would place a writing pen into its cap.

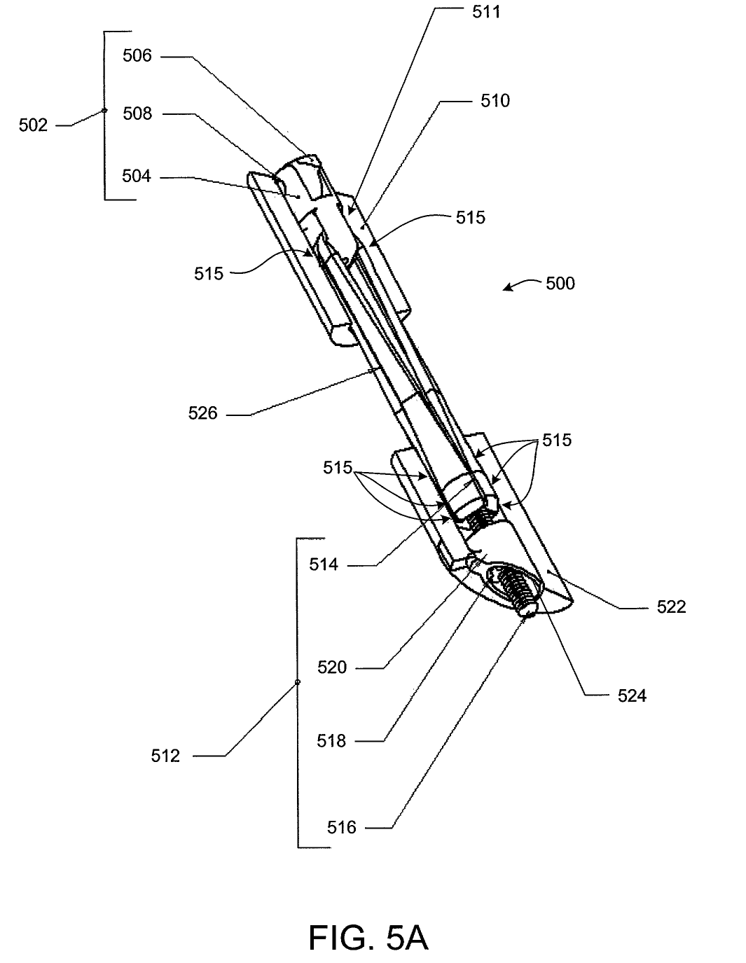

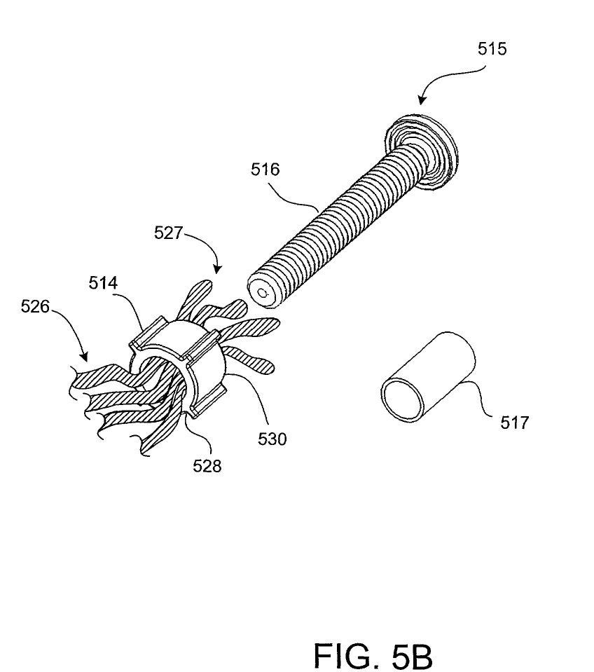

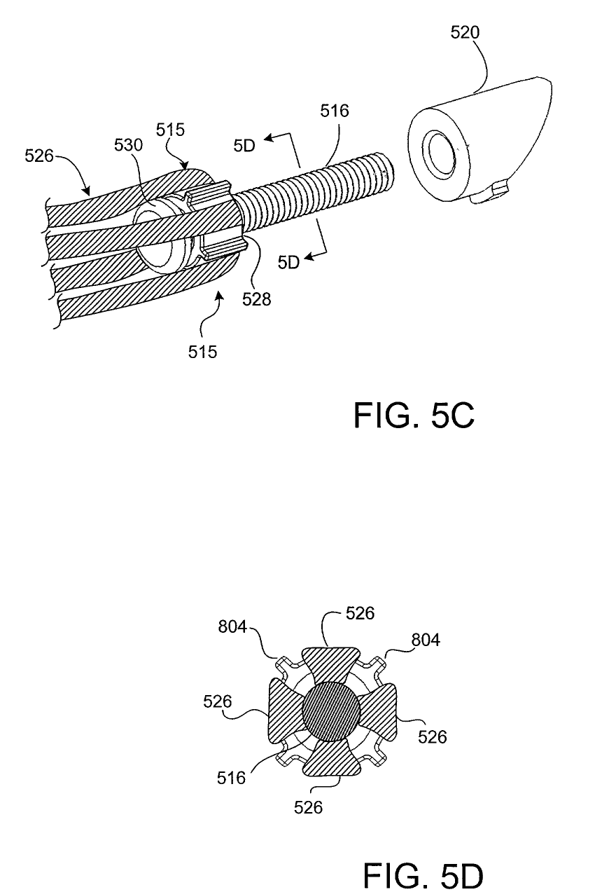

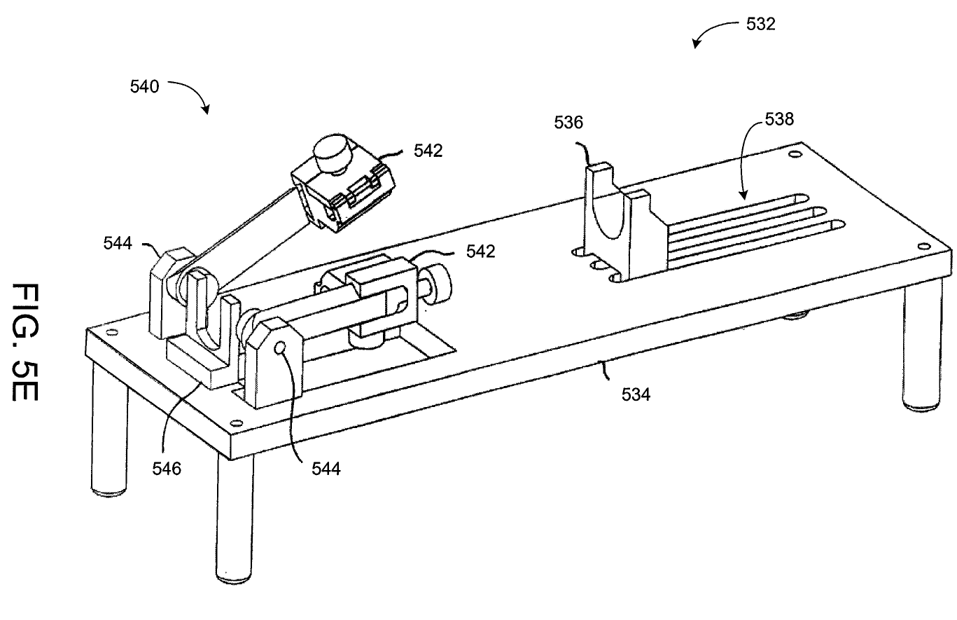

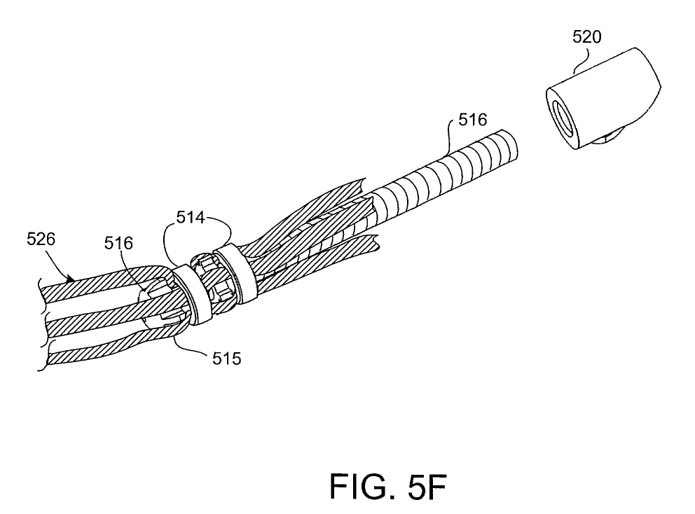

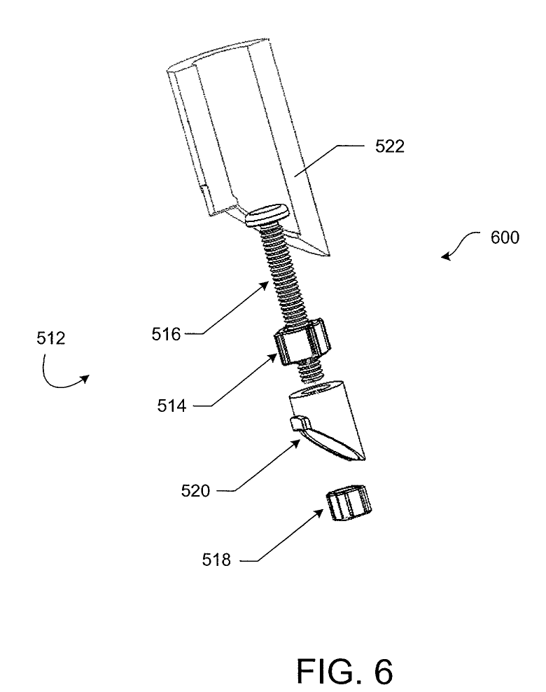

FIG. 5A shows an isometric assembly view of another variation of the system that does not necessarily include a tooling rod. It, nevertheless, may be installed from a single end of the bone tunnel. As seen in this variation (500), femoral or distal assembly (502) may be an assembly similar to that described above and is anchored at the femoral cortex (510). Generally, tibial assembly (512) is anchored at the tibial cortex (522) with graft (526) securely extending between the femoral assembly (502) and tibial assembly (512). Furthermore, this assembly (500) may be fully assembled ex vivo and inserted within a patient as a completed unit. Once placed within the patient, the device may simply be adjusted to attain the optimal tension within grafts (526). The device of variation (500) may be initially positioned within a pre-bored bone tunnel. This bone tunnel may be drilled linearly through tibial cortex (522) and femoral cortex (510).

A calibrated depth probe may optionally be inserted within the tunnel to measure the overall length of the drilled tunnel and the length from the tibial spine to the tibia cortex prior to placement of the assembly (500) within. Based upon the measured lengths, the effective length of the graft (526) may be adjusted accordingly, taking into account the lengths of the components of assembly (500).

Femoral assembly (502), as above, may be comprised of femoral component (504). Component (504) is generally a fixation member which may house pivotable anchor member (506) at a distal end and which may define an opening or loop (511) at a proximal end of component (504). Pivotable anchor member (506) preferably rotates freely about pivot (508) to enable a secure engagement at femoral cortex (510). To facilitate the placement of femoral assembly (502) at femoral cortex (510), a guidewire (not shown) may be advanced through both femoral tunnel (510) and tibial tunnel (522). One end of the guidewire may be releasably attached to femoral assembly (502) or both femoral (502) and tibial (512) subassemblies; the guidewire may then be simply pulled through the bone tunnel to advance assembly (500) into position. Once femoral assembly (502) is anchored to femoral cortex (510) the guidewire may then be removed. The graft (526) is looped through opening (511) of component (504) and is secured at tibial assembly (512), as described below. Alternatively, graft (526) may be fixed to component (504) through other mechanical fastening methods, e.g., graft (526) may be crimped, sutured, or fixed via tines, etc.

In this variation (500), tibial assembly (512) may generally be comprised of a length and tension adjuster, e.g., a screw or bolt (516) that is adjustably secured to tibial seat (520) along the shank of bolt (516) with fastener (518). Tibial seat (520) is shown as having a sloped surface (524) and is described in further detail below. Fastener (518) used may be any adjustable fastening device, e.g., a nut. The terminal ends of graft (526) are preferably secured between the shank of bolt (516) and at least one ring fastener (514) which is slidably adjustable along bolt (516) during graft (526) placement.

As shown, when tibial assembly (512) is positioned within the pre-bored bone tunnel, the assembly (512) is positioned such that a presentation surface positions graft (526) against the walls of the bone tunnel. Presentation surfaces (515) are configured such that when tibial assembly (512) has been seated, the outer surface of ring fastener (514) or the head of bolt (516) as well as component (504), gently presses grafts (526) against the walls of the bone tunnel and are separated from where the terminal ends of graft (526) are secured between bolt (516) and ring fastener (514). This independent area of graft (526) to bone contact generally promotes the healing of graft (526) to the bone to ultimately result in a strong biological construct.

The length and tension adjuster (516), is described here generally, and it may include helical threads allowing for simple and predictable adjustment of the length of the graft and the tension of the graft after introduction and anchoring of the system. The mechanical concept is to pull on adjuster (516) until the proper tension is achieved and then lock the adjuster in place. Here, the adjustment and adjuster (516) are locked in place by the nut. Other adjuster configurations based upon-that concept are apparent: for instance, shafts that are pulled and fixed in place by “grab” rings, cross pins, cotter pins, swaged stops, or the like.

One example of securing the grafts (526) is shown in FIGS. 5B to 5D. FIG. 5B illustrates an example for securing grafts (526) prior to assembly. Once grafts (526) have been looped through or fixed to femoral assembly (502) and through ring (514), grafts (526) may be pre-tensioned approximately to a predetermined load relative to femoral assembly (502) using, e.g., a variation of a pre-tensioning station as shown in FIG. 5E. As seen, assembly (532) comprises a platform (534) having adjustable jigs placed upon its surface. First jig (536) may be used to position the femoral assembly (502) with grafts (526) extending from first jig (536) to second jig assembly (540). The terminal ends of grafts (526) may be placed and securely held within graft attachments (542), which are positioned at the ends of lever arms and which may be adjustably pivoted about pivots (544). The terminal ends of the grafts (526) may be extended through graft attachments (542) and attached to the pre-stretching weights (not shown). Jig (546) may be used as a guide for positioning the tibial seat (520), or it may also be used as a guide for the weights. Once the grafts (526) have been desirably positioned, first jig (536) may be repositioned within adjustment slots (538) to adjustably provide for the desired amount of stretch in each of the grafts (526).

With grafts (526) in the stretched configuration (weights are omitted for clarity), within ring (514), as in FIG. 5B, the ring (514) is slid to a desirable position along grafts (526), as may be determined by the depth probe. Ring (514) defines an opening preferably having a diameter which is large enough to accommodate bolt (516) and a number of graft (526) segments, i.e., at least one graft (526) and preferably two or more grafts (526). Adjuster (516) may have a nominal root diameter of about 0.16 in. and a shank length of about 1.2 in. In one variation as shown in FIG. 5B, with grafts (526) positioned within ring (514), adjuster (516) may be pushed through the opening defined through ring (514) such that grafts (526) are wedged or pressed between the inner surface of ring (514) and adjuster (516). As shown, the adjuster (516) may be threaded along a portion of its shank, and if so, a sleeve or sheath (517) may initially be placed over adjuster (516) prior to insertion to prevent damage to grafts (526) during adjuster (516) insertion. Once adjuster (516) has been situated within ring (514), in that grafts (526) are securely positioned against ring (514), sleeve (517), if used, may then be removed. Sleeve (517) may be any type of biologically inert material, e.g., plastic, stainless steel, etc.

The adjuster (516) may be inserted through ring (514) from a second side (530), where terminal ends (527) of graft (526) extend, to first side (528), as seen in FIG. 5B. After placement, the assembly of the adjuster (516), with grafts (526) pressed against ring (514), may then be inverted so that grafts (526) are now positioned on the outside surface of ring (514), as shown in FIG. 5C. Adjuster (516) may then be positioned within tibial seat (520), for placement within the knee. When inverted, grafts (526) are preferably spaced evenly about ring (514). As shown in FIG. 5D, which is an end view of grafts (526) positioned between ring (514) and adjuster (516), grafts (526) may be evenly spaced apart circumferentially depending upon the number of grafts used. The surfaces between the ring (514) and the adjuster (516) are the point of fixation of grafts (526). This mechanical fixation holds the graft in place until biological fixation occurs distal to the area of mechanical fixation, thus replacing mechanical fixation. Due to the fact that each component of anchoring, tissue fixation, and tissue healing require unique parameters for optimal benefit, this system allows for independent control of each of the components. This creates significant flexibility within the system, and eliminates conflicting forces that otherwise exist when said components are not independent, e.g., pressure required for fixation may be supra-optimal for tissue healing; point of strongest anchoring, cortex, may not be ideal for healing.

The inversion of the grafts (526) in the depicted embodiment and the subsequent introduction of the system into the bone tunnel “presents” the surface of those grafts (526) to the interior of the bone tunnel. In doing so, the outer surface of ring (514) and head of adjuster (516) create a presentation surface to position grafts (526) for healing against the bone. Alternatively, if ring (514) is not inverted, the tibial subassembly including one or more rings (514) may appear as shown in FIG. 5F. In this variation, the grafts (526) are not external to rings (514), so the head of adjuster (516) creates the presentation surface (515).

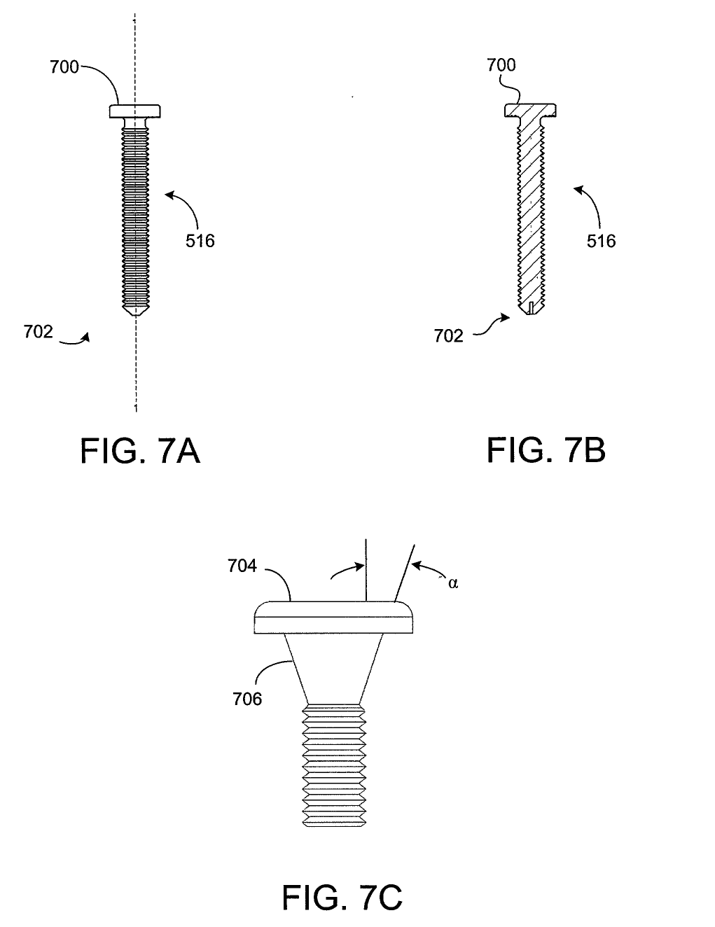

FIG. 6 shows an exploded assembly view (600) of tibial assembly (512) in which grafts (526) have been omitted for clarity. Fastener or nut (518) may be placed over adjuster (516). Grafts (526) are preferably placed in a tensioned condition in the knee, secured between tibial assembly (512) and femoral assembly (502. The act of tightening (or loosening) adjuster (516) with relation to tibial seat (520) may be accomplished by using an optional tool, as described below. In adjusting the tension of the grafts, the adjuster (516) is pulled through tibial seat (520) until a proper tension on grafts (526) is achieved. Once the proper tension is achieved, the fastener (518) is positioned to fix adjuster (516) in place and installation of the system may be complete. The tension in grafts (526) may be re-adjusted intra-operatively as desired after installation is complete. This system may further allow for re-adjustment for a short period of time post-operatively.

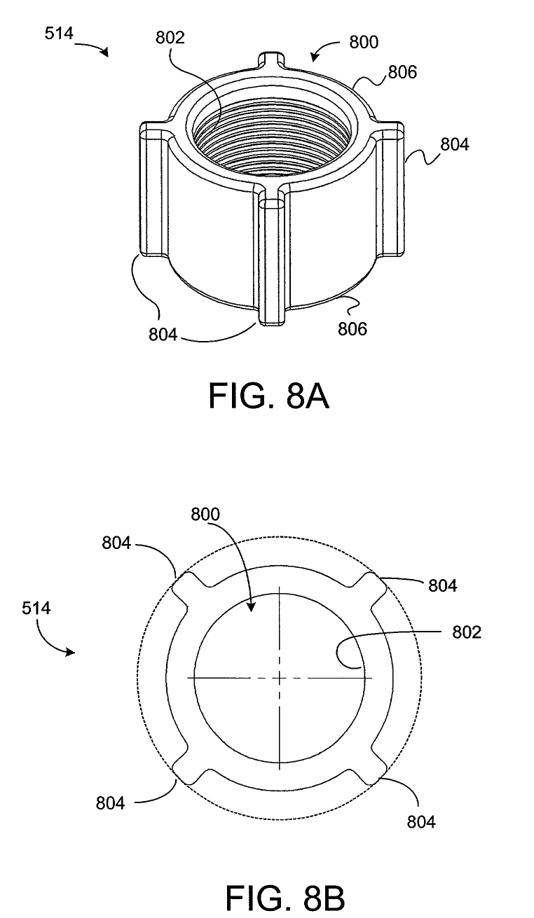

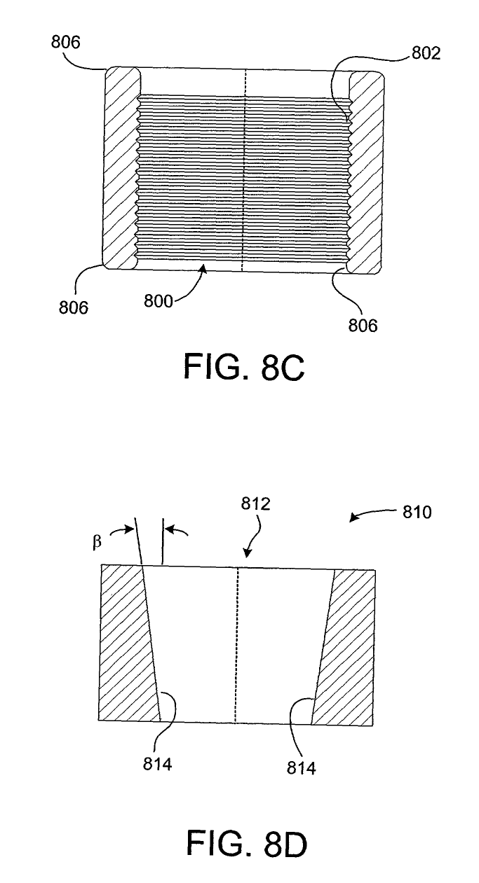

FIGS. 7A and 7B show side and cross-sectional views, respectively, of adjuster (516). Adjuster (516) may be made of a biocompatible material, e.g., stainless steel, titanium, polymers, etc., or it may be made entirely of a bioabsorbable material, as described above. Furthermore, although shown with threading along the shank of the adjuster (516), the threading may be omitted from the shank near the head (700) of adjuster (516) to provide a smooth surface for contact against grafts (526). Furthermore, tip (702) may be blunt, as shown, or it may be tapered.

Moreover, to optimize and distribute the loading generated by the tensioned grafts (526) against the adjuster (516), a portion of the adjuster may optionally be tapered or otherwise varied, as shown in FIG. 7C. The adjuster variation is shown as having a tapered section (706) adjacent to head (704). Tapered section (706) may be tapered at an angle, α, to aid in reducing the stress concentration in the tibial assembly by distributing the stress over a larger interface between the ring and the tapered bolt. Alternatively, the adjuster (514) may be configured with protrusions or other geometric variations, as shown in FIG. 5F, to optimize and distribute loads generated by the tensioned grafts (526). To complement a tapered bolt section, the ring may also be tapered or otherwise varied along its inner diameter, as described below.

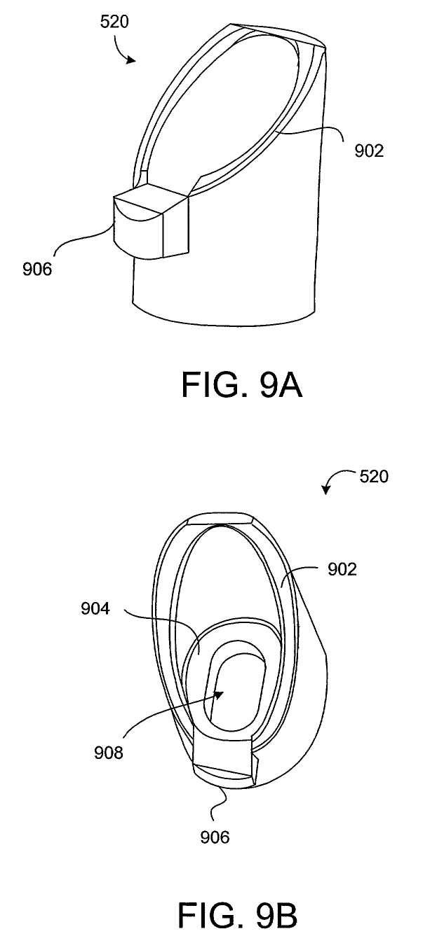

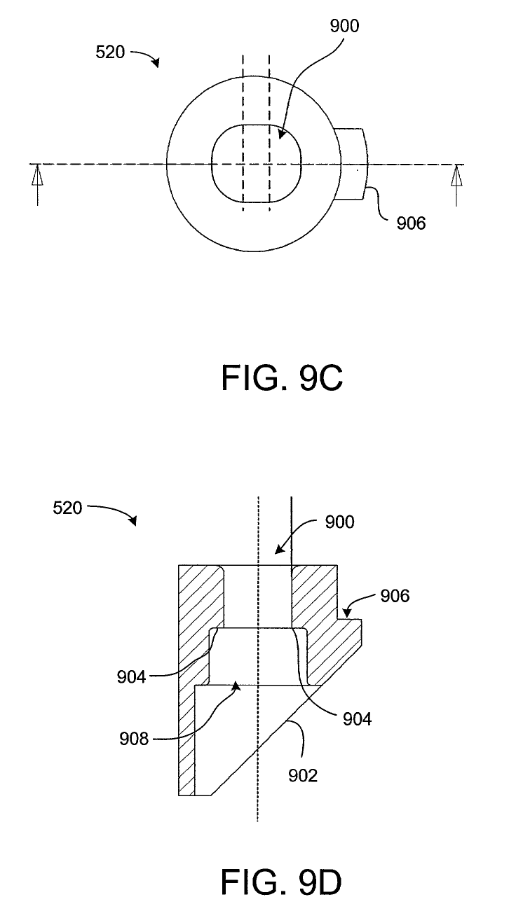

FIGS. 8A to 8C show isometric, top, and cross-sectioned side views, respectively, of one variation of ring (514). As seen in FIG. 8A, ring (514) defines opening (800). The inner surface (802) of ring (514) may be threaded, as shown in this variation, or it may also present a smooth surface. Alternatively, inner surface (802) may also be roughened to aid in gripping grafts (526) wedged against inner surface (802). The lip (806) of ring (514) may be radiused or otherwise smoothed to present a surface that does not damage the grafts (526). Furthermore, a number of projections (804) may be defined along the outer surface of ring (514), as mentioned above, to aid in handling ring (514) as well as in separating grafts (526). Four projections (804) are shown in this variation; however, any number of projections (804) may be used depending upon the number of grafts utilized. The projections may also be in various other forms, e.g., notches, raised nubs, rounded edges, etc. Alternatively, projections (804) may also be omitted entirely from ring (514).

Ring (810) may also have an inner surface (814) which is tailored to also facilitate the distribution of tensile loads and to reduce stress concentrations generated in the grafts. As shown, the inner surface of ring (810) may be tapered at an angle, β, or otherwise varied, through opening (812). This tapered surface (814) may be angled to complement the tapered section (706) of the bolt shown in FIG. 7C. Alternatively, tapered surface (814) may be implemented alone without a tapered bolt, or the tapered bolt may be implemented alone, or both the tapered bolt and the complementary tapered surface (814) may be implemented together. The desired geometry of the inner surface of ring (810) may be achieved by a number of methods including, but not limited to, injection molding, casting, machining, forging or intra-operatively swaging or deforming ring (810).

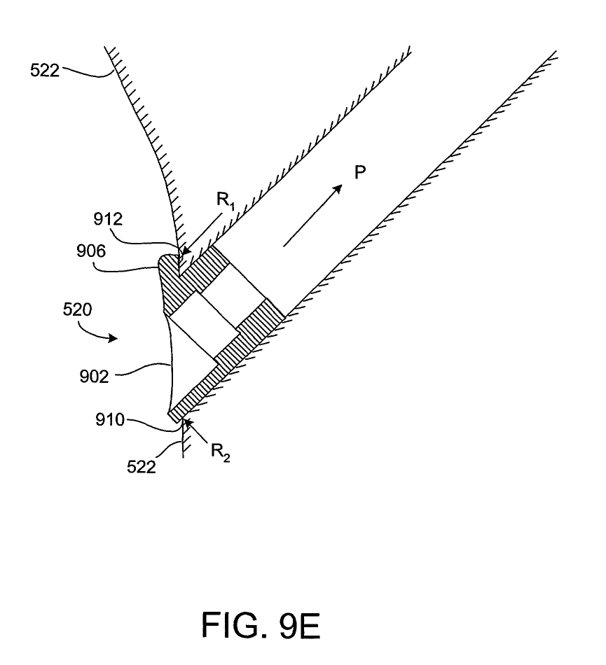

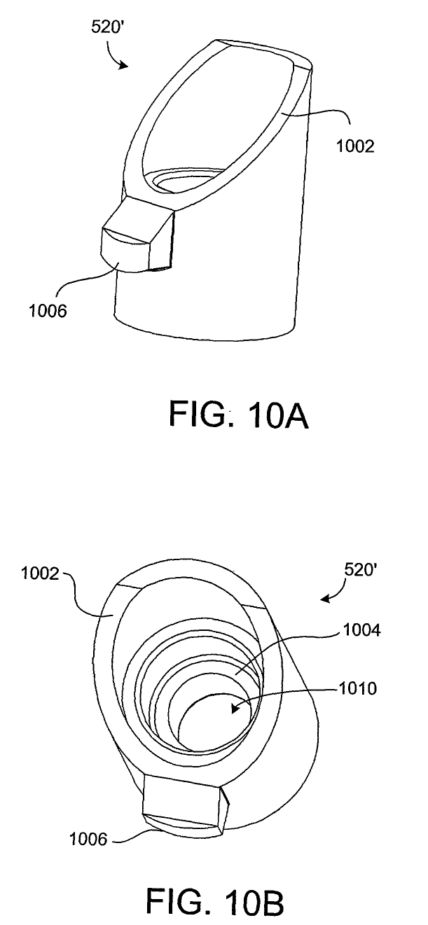

FIGS. 9A and 9B show various isometric views of one variation of tibial seat (520). As shown, tibial seat (520) is generally a tubular shaped member having a sloped surface (902) at a first end and an engagement surface (904) at a second end. The sloped surface typically approximates the surface of the exterior opening of the bone tunnel into which it is placed. Tibial seat (520) may have an outer diameter of, e.g., about 0.4 in. and an overall length of, e.g., about 0.6 in. Receiving channel (908) is defined through engagement surface (904) and it may be formed in a variety of shapes, shown in this variation as an elliptically-shaped opening and having dimensions of approximately 0.22 by 0.17 in. These sizes (and the others mentioned in this description) are for informational or relational purposes only and are not critical to the use of the system. The choice of appropriate sizes is well within the skill of a designer in this industry.

FIGS. 9C and 9D, which show bottom and cross-sectioned side views of a tibial seat respectively, show channel (908) and distal opening (900) as defined in the bottom surface. Sloped surface (902) may be sloped at various angles, shown here at a 45° angle. Tibial stop (906) is also seen as a projection extending from the outer surface of tibial seat (520). Sloped surface (902) allows tibial seat (520) to be installed nearly flush against the surface of the cortical bone as well as providing other advantages, as described further below. Tibial stop (906) extends radially from the the tibial seat (520) to prevent the seat from sliding wholly within the bone tunnel. The tibial stop (906) may extend out, e.g., about 0.06 in. from the outer surface of seat (520) and may be formed integrally with seat (520) from the same material, which may comprise any of the bioabsorbable materials described above or any of the biocompatible, plastic, or metallic materials described herein. Tibial stop (906) is preferably located at the lower end of sloped surface (902) and is adapted to contact the tibial cortex (522) and prevent the longitudinal dislodgement of tibial seat (520) when grafts (526) have been tightened. The use of a single stop located as shown helps to register the tibial seat (520) with the bone tunnel. As alternatives, a number of tibial stops (906) may also be formed to surround the entire circumference about tibial seat (520) or it may be formed selectively about the circumference with multiple individual projections. These designs lack the registering capability and may be harder to fit in some anatomies.

Furthermore, as mentioned above, when tibial stop (906) is but a single projection positioned opposite of the upper end of the sloped surface (902), the tibial seat (520) is a self-centering and self-seating device within the bone, as shown in FIG. 9E. As also shown, when the tensile loading, P, is presented by the grafts, tibial seat (520) becomes configured to automatically seek the lowest energy state in providing a stable platform for graft fixation. As seat (520) is pulled against tibial cortex (522), it contacts the bone at contact region (912) against tibial stop (906) and at contact region (910) against the upper end of sloped surface (902). Tibial stop (906) preferably provides the entire reaction load (or at least a majority of the reaction load), R₁, against the tensile load, P. To counter the moment generated between R₁ and P, the upper end of sloped surface (902) preferably reacts to provide a reaction load, R₂, to help stabilize the tibial seat (520) against the tibial cortext (522). Thus, the entire force to counter the tensile load, P, may be distributed entirely between two contact areas or regions (910), (912) which provides for stable anchoring. Moreover, the tibial seat (520) is configured such that it presents a low profile by remaining flush against the bone surface and does not protrude a significant distance, if any, beyond the tibial cortext (522). Furthermore, because of the sloped surface (902) and positioning of tibial stop (906), tibial seat (520) can remain flush against the tibial cortex (522) regardless of the particular variances in bone geometry between different patients.

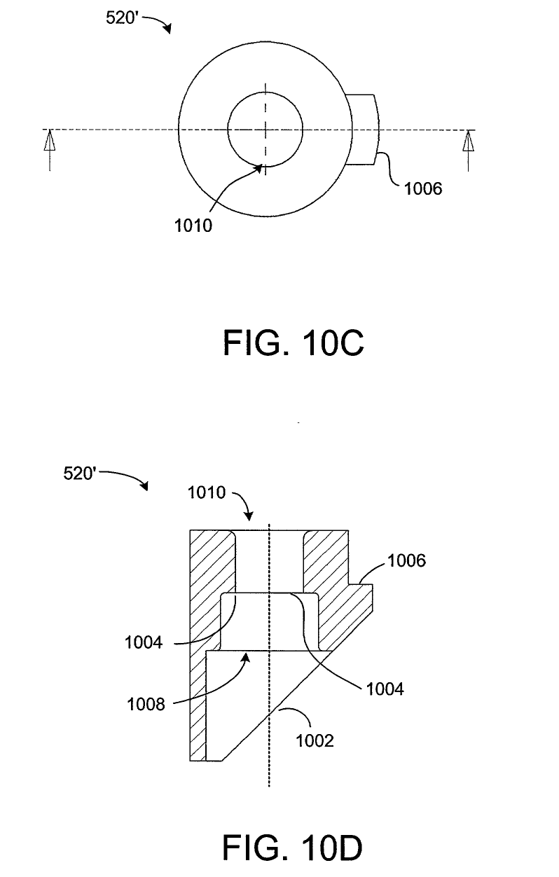

FIGS. 10A and 10B show various isometric views of another variation of tibial seat (520′). This variation (520′) likewise forms sloped surface (1002) with tibial stop (1006) at the apex of surface (1002). FIGS. 10B and 10D, which is a cross-sectioned side view, show channel (1008) extending through seat (520′). Yet variation (520′) defines a circularly-shaped channel opening (1010), as seen in FIG. 10C. As further seen, engagement surface (1004) may be formed at various levels. For instance, depending upon the size of the fastener used, one having a smaller diameter may be utilized to mate against engagement surface (1004); alternatively, a fastener having a larger diameter may be used with the same tibial seat (520′) but rest against another engagement surface defined above engagement surface (1004), as shown in FIG. 10D.

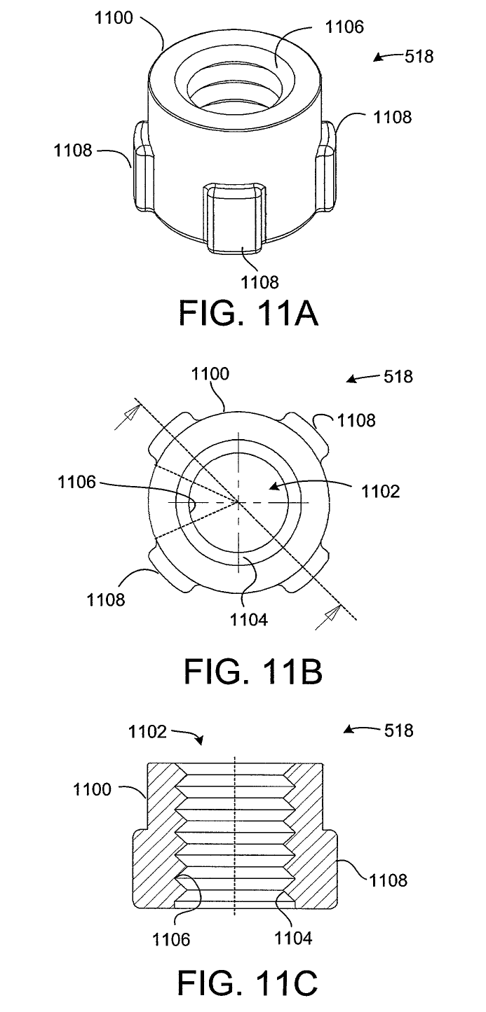

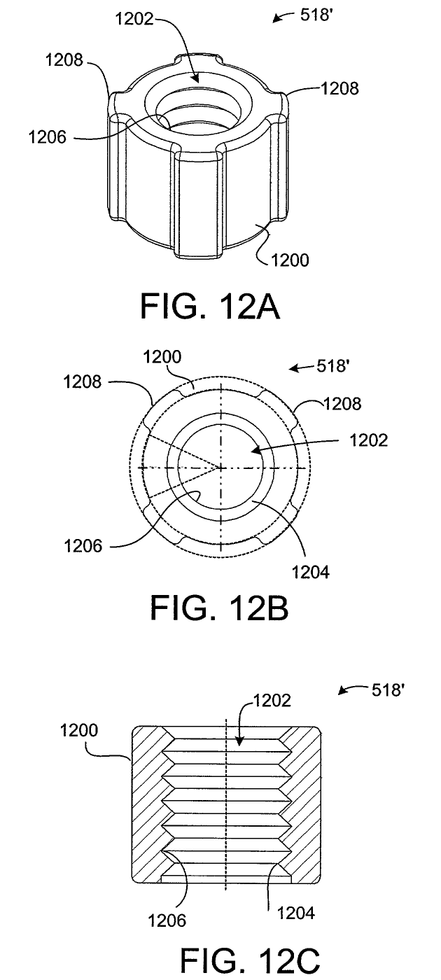

FIGS. 11A to 11C show isometric, top, and cross-sectioned side views, respectively, of one variation of fastener (518). As seen, fastener (518) may be formed by a cylindrical member (1100) having a diameter of, e.g., about 0.24 in., and a height of, e.g., about 0.2 in. Fastener (518) defines a receiving channel (1102) therethrough for securely receiving adjuster (516). Inner surface (1106), as seen in FIGS. 11B and 11C, is preferably threaded (1104) and channel (1102) has an appropriate diameter to correspondingly mate with adjuster (516). Projections (1108) may be defined about the outer surface of member (1100) to facilitate the handling and rotation of fastener (518). The projections (1108) are shown to have a height of about half of the height of member (1100), but they may be formed along the entire height of member (1100), as shown in FIG. 12A. As mentioned above, fastener (518) may be a nut, which secures onto the adjuster (516); alternatively, the fastener may also be a deformable fastener, which clips onto the distal end of the bolt to secure it, e.g., a U-Nut, swaged fitting, etc.

FIGS. 12A to 12C show isometric, top, and cross-sectioned side views, respectively, of another variation of fastener (518′). Fastener variation (518′) is similarly formed by cylindrical member (1200), which defines a receiving channel (1202) having an inner surface (1206) and threading (1204). But projections (1208) are shown to extend along the entire height of the member (1200) to facilitate handling and fastening of the device against tibial seat (520). Any number of projections (1208) may be utilized in this variation (518′) or variation (518) depending upon the desired results and application.

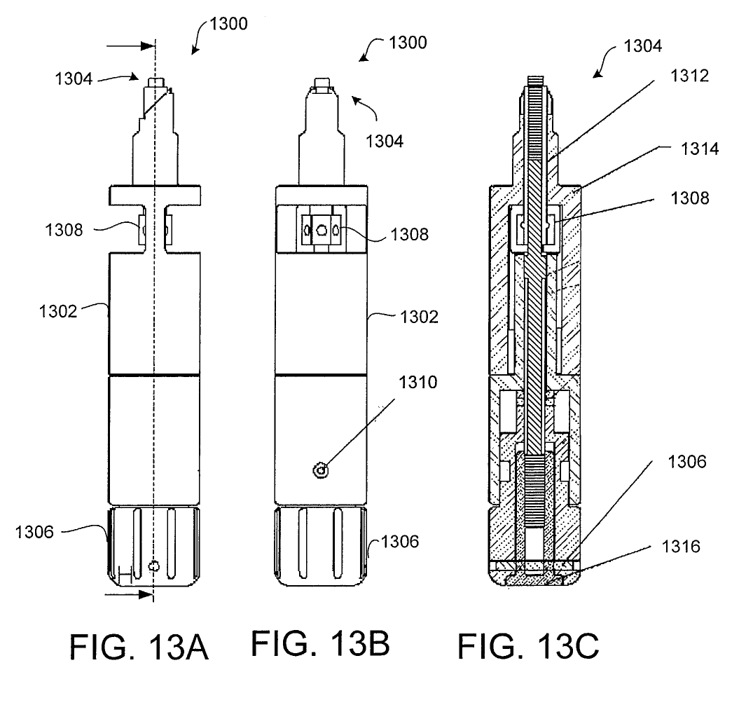

FIGS. 13A and 13B show side views of fastening tool (1300), which may be used to secure fastener (518) against tibial seat (520) when tightening or loosening grafts (526). Tool (1300) maybe generally comprised of a body (1302) having an engagement tip (1304) at a distal end and a adjuster engagement control (1306) at a proximal end. A fastener engagement control (1308) may also be included within tool (1300) for tightening or loosening fastener (518). In use, once the femoral (502) and tibial assemblies (512) have been anchored on the femoral cortex (510) and tibial cortex (522), respectively, engagement tip (1304) of tool (1300) may be mated against the exposed distal end of adjuster (516) and tibial seat (520). As seen in FIG. 13C, which is a cross-sectioned side view of tool (1300) from FIG. 13A, tool (1300) preferably has a central shaft (1312) rotatably held within exterior (1314) and connected to adjuster engagement control (1306). Central shaft (1312) may be partially threaded near its distal end for releasably engaging the distal end of adjuster (516). The proximal end of central shaft (1312) may also be partially threaded for connection to control (1306) via attachment (1316).

It is generally preferable that when fastener (518) is adjusted, i.e., tightened or loosened against tibial seat (520), that adjuster (516) is prevented from rotating about its own longitudinal axis in order to prevent grafts (526) from twisting. Thus, when engagement tip (1304) is mated against adjuster (516) and tibial seat (520), control (1306) is preferably rotated to engage the distal end of adjuster (516). Once engaged, control (1306) may be desirably rotated to engage and incrementally advance adjuster (516) towards tool (1300). Adjuster (516) may be advanced a distance to provide clearance between fastener (518) and surface (904) of tibial seat (520). The pulling of adjuster (516) will accordingly increase the tension in grafts (526). Fastener engagement control (1308), which preferably mates with fastener (518) at the distal end of control (1308) may be rotated manually or automatically, to accordingly tighten or loosen fastener (518) to lock or unlock the adjuster position. When the tightening (or relaxing) procedure has been completed, control (1306) may then disengage from adjuster (516). This adjustment procedure may be repeated as necessary adjuster post-deployment or post-operatively and allows for the tensioning or relaxing of grafts (526) without the need for removing the entire assembly.

Although not specifically shown in the variation of the device shown in the Figures, it is within the scope of this description to include in the tool (1300) a device for measuring the tension in the graft, e.g. load cell, spring scale, strain gage, etc., with regard to the tibial seat. A display for the resulting tension may be included as well.

In describing the system and its components, certain terms have been used for understanding, brevity, and clarity. They are primarily used for descriptive purposes and are intended to be used broadly and construed in the same manner. Having now described the invention and its method of use, it should be appreciated that reasonable mechanical and operational equivalents would be apparent to those skilled in this art. Those variations are considered to be within the equivalence of the claims appended to the specification.

We claim as our invention:

1. An ACL reconstruction system, insertable as an assembly into a pre-bored tunnel passing through a tibia and a femur and anchorable at both ends, the system comprising:
a.) at least one graft member extending from a distally located subassembly to a proximally located subassembly,
b.) the distally located subassembly configured to fixate the at least one graft member, and
c.) the proximally located subassembly comprising a graft fixation surface adapted to hold the at least one graft member to said proximally located subassembly, and a presentation surface separated from the graft fixation surface, said presentation surface configured to position the at least one graft member against the wall of the pre-bored tunnel.

2. The system of claim 1 where the system is insertable into the tunnel as a complete assembly having at least one anchoring element for anchoring the system to bone.

3. The system of claim 1 where the system is insertable through one end of the pre-bored tunnel.

4. The system of claim 1 where the distal subassembly includes an anchor to the bone.

5. The system of claim 1 where the proximal subassembly includes an anchor to bone.

6. The system of claim 1 where the distal subassembly includes an anchor to the bone with a pivotal member.

7. The system of claim 6 where the pivotable member comprises a pawl.

8. The system of claim 1 where the distally located subassembly comprises a winged anchoring toggle configured to engage the end of the pre-bored tunnel.

9. The system of claim 1 where the at least one graft member is secured to and extends from the proximally located subassembly, loops through the distally located subassembly, and extends to and is secured to the proximally located subassembly.

10. The system of claim 9 where the at least one graft member comprises exactly one graft member.

11. The system of claim 9 where the at least one graft member comprises two graft members.

12. The system of claim 1 where the at least one graft member comprises exactly one graft member having a first end and a second end, the first end being fixed to the distal subassembly and the second end being fixed to the proximal subassembly.

13. The system of claim 1 where the graft fixation component of the proximally located subassembly comprises a ring-like member with an open center and an adjuster passing through said open center and adapted to hold the at least one graft member between the ring-like member and the adjuster.

14. The system of claim 13 where the adjuster comprises a helically grooved member.

15. The system of claim 13 where the adjuster comprises a helically grooved member passing through said ring-like member open center, the helically grooved member and said ring-like member forming the graft fixation surface.

16. The system of claim 13 where the ring-like member has a size and an outer surface forming the presentation surface.

17. The system of claim 13 where the adjuster comprises a presenting surface separate from the fixation surface for positioning the graft member against the bone.

18. The system of claim 13 where the graft member is protected by a sleeve disposable over the adjuster during assembly.

19. The system of claim 2 where the anchoring element of the proximal subassembly, the distal subassembly, or both comprises a substantially cylindrical member adapted to fit partially into the tunnel and having at least one protuberance to secure the anchor component to the bone.

20. The system of claim 13 where the adjuster is adjustably attachable to an anchor component.

21. The system of claim 13 where the adjuster is adjustably attachable to an anchor component by a locking member.

22. The system of claim 1 further comprising a tension measuring component configured to measure tension on the at least one graft member.

23. The system of claim 22 further comprising a tension indicator for displaying tension values on the at least one graft member.

24. The system of claim 1 wherein the distally located subassembly comprises a material selected from the group consisting of biocompatible metals, biodegradable polymers and their copolymers, and ceramics.

25. The system of claim 24 wherein the biocompatible metals comprise stainless steel, titanium, or nickel-titanium alloys.

26. The system of claim 24 wherein the biodegradable polymers and their copolymers are selected from the group consisting of monomers comprising esters, anhydrides, orthoesters, amides, polyglycolide, polylactide, poly-α-caprolactone, polydioxanone, polyglyconate, copolymers of polylactide and polyglycolide, hydroxyapetite, and the block and random copolymers of these polymers.

27. The system of claim 1 wherein the proximally located subassembly comprises a material selected from the group consisting of biocompatible metals, biodegradable polymers and their copolymers, and ceramics.

28. The system of claim 27 wherein the biocompatible metals comprise stainless steel, titanium, or nickel-titanium alloys.

29. The system of claim 27 wherein the biodegradable polymers and their copolymers are selected from the group consisting of monomers comprising esters, anhydrides, orthoesters, amides, polyglycolide, polylactide, poly-α-caprolactone, polydioxanone, polyglyconate, copolymers of polylactide and polyglycolide, hydroxyapetite, and the block and random copolymers of these polymers.

30. An ACL reconstruction system with at least one graft member, insertable into a pre-bored tunnel passing from a tibia to a femur, and having an adjustable tension after said insertion comprising:
a.) at least one graft member extending from a distally located subassembly to a proximally located subassembly,
b.) the distally located subassembly configured to fixate the at least one graft member, and
c.) the proximally located subassembly having:
i.) engaging surfaces configured to engage an adjustment tool and to adjust tension in the at least one graft member after insertion into the tunnel, and
ii.) graft fixation surface.

31. The system of claim 30 where the proximally located subassembly further comprises an anchoring component configured to anchor the proximally located anchoring and graft fixation subassembly in the proximal end of the pre-bored tunnel.

32. The system of claim 30 where the system is insertable into the tunnel as a complete assembly having at least one anchoring element for anchoring the system to bone.

33. The system of claim 30 where the system is insertable through one end of the pre-bored tunnel.

34. The system of claim 30 where the distally located subassembly comprises at least a pivotable member.

35. The system of claim 30 in combination with the adjustment tool, the adjustment tool being configured to move the engagement surfaces and to adjust tension on the at least one graft member.

36. The system of claim 31 where the proximally located subassembly further comprises an adjuster movably attached but fixable to the anchoring component.

37. The system of claim 30 where the proximally located subassembly comprises a ring-like member with an open center and an adjuster passing through said open center and adapted to hold the at least one graft member between the ring-like member and the adjuster.

38. The system of claim 37 where the adjuster comprises a helically grooved member.

39. The system of claim 37 where the adjuster comprises a helically grooved member screw or bolt passing through said ring-like member open center and through the anchor, the screw or bolt having a head opposite the anchor and forming the the graft fixation surface between the ring-like member and the adjuster head.

40. The system of claim 37 where the ring-like member has a size and an outer surface forming a presentation surface.

41. The system of claim 37 where the adjuster comprises a helically grooved member passing through said ring-like member open center, the helically grooved member and said ring-like member forming the graft fixation surface.

42. The system of claim 37 where the graft member is protected by a sleeve disposable over the adjuster during assembly.

43. The system of claim 31 where the anchoring component comprises a substantially cylindrical member adapted to fit partially into the tunnel and having at least one protuberance to anchor the anchor component to the tunnel.

44. The system of claim 43 where the adjuster is adjustably attachable to the anchoring component.

45. The system of claim 43 where the adjuster is adjustably attachable to the anchor component by a nut.

46. The system of claim 30 further comprising a tension measuring component configured to measure tension on the at least one graft member.

47. The system of claim 30 further comprising a tension indicator for displaying tension values on the at least one graft member.

48. The system of claim 35 where the adjustment tool further comprises an integral tension indicator configured to indicate tension in the at least one graft member both during adjustment of that tension and before and after that adjustment.

49. The system of claim 37 where the anchor component comprises a substantially cylindrical member adapted to fit partially into the tunnel and having at least one protuberance to anchor the anchor component to the bone.

50. The system of claim 49 where the adjuster is adjustably attachable to the anchor component.

51. The system of claim 30 where the proximally located subassembly engaging surfaces are configured to move the graft anchor upon movement of the engaging surfaces and to adjust the tension of the at least one graft member.

52. The system of claim 30 where the proximally located subassembly is configured to allow measurement of the tension on the at least one graft member.

53. The system of claim 30 where the distally located anchoring subassembly is configured to be affixed to the femoral end of the tunnel.

54. The system of claim 30 where the proximally located subassembly is configured to be affixed to the tibial end of the tunnel.

55. The system of claim 39 where the proximally located subassembly is configured to allow measurement of the tension of the graft member throughout the rotation of a knee having an inserted system in the tunnel.

56. A subassembly for reconstructing an ACL in a knee having a pre-bored tunnel passing from a tibia to a femur and adapted, independently, to promote fixation of at least one graft member to the bone tunnel and to fix the subassembly in the bone tunnel, comprising:
an anchoring component to fix the subassembly in the bone tunnel and having a size selected to slide partially into the bone tunnel and having at least one protuberance extending from the anchoring component's exterior to prevent the anchoring component from completely entering the tunnel.

57. The subassembly of claim 56 further comprising a graft fixation component supported by but separated from the anchoring component and configured to fix the at least one graft member ex vivo.

58. The subassembly of claim 57 further comprising an adjuster configured to allow adjustable variation of the distance between the graft fixation component and the anchoring component.

59. The subassembly of claim 57 further comprising an adjuster configured to allow adjustable variation of the distance between the graft fixation component and the anchoring component.

60. The subassembly of claim 59 where the adjuster is configured to allow adjustment of the distance between the graft fixation component and the anchoring component after installation from outside the body.

61. The subassembly of claim 57 wherein the graft fixation component is adapted to fix said at least one graft member between two subcomponents of the graft fixation component.

62. The subassembly of claim 57 where the graft fixation component comprises a ring-like member with an open center and an adjuster passing through said open center and adapted to hold the at least one graft member between the ring-like member and the adjuster.

63. The subassembly of claim 62 where the adjuster comprises a helically grooved member.

64. The subassembly of claim 62 where the adjuster comprises a helically grooved member screw or bolt passing through said ring-like member open center and through the anchor, the screw or bolt having a head opposite the anchor and forming the the graft fixation surface between the ring-like member and the adjuster head.

65. The subassembly of claim 64 where the ring-like member has a size and an outer surface forming a presentation surface.

66. The subassembly of claim 62 where the adjuster is adjustably attachable to the anchoring component.

67. The subassembly of claim 66 where the adjuster is adjustably attachable to the anchor component by a nut.

68. The subassembly of claim 56 further comprising a tension measuring component configured to measure tension on the at least one graft member.

69. The subassembly of claim 62 further comprising a tension indicator for displaying tension values on the at least one graft member.

70. An ACL reconstruction system kit, combinable with at least one graft member to form an ACL reconstruction system, completely insertable into one end of a pre-bored tunnel passing from a tibia to a femur and fixable in said tunnel, the system comprising:
a.) a distally located anchoring subassembly configured to anchor the system to the distal end of the tunnel, and
b.) a proximally located graft fixation and anchoring subassembly comprising an anchor, a graft fixation surface adapted to hold the at least one graft member in said graft fixation and anchoring subassembly, and a presentation surface separated from the graft fixation surface and from the anchor, said presentation surface configured to press the at least one graft member into the wall of the pre-bored tunnel after installation in that tunnel.

71. The system of claim 70 where the distally located anchoring subassembly is configured to be affixed to the femoral end of the tunnel.

72. The system of claim 70 where the proximally located anchoring and graft fixation subassembly is configured to be affixed to the tibial end of the tunnel.

73. The system of claim 70 where the distally located anchoring subassembly comprises a pivotable member.

74. The system of claim 73 where the pivotable member comprises a pawl.

75. The system of claim 70 where the distally located anchoring subassembly comprises a winged toggle configured to engage the end of the pre-bored tunnel.

76. The system of claim 70 where the at least one graft member is secured to and extends from the proximally located anchoring and graft fixation subassembly, loops through the distally located anchoring subassembly, and extends to and is secured to the proximally located anchoring and graft fixation subassembly.

77. The system of claim 76 where the at least one graft member comprises exactly one graft member.

78. The system of claim 76 where the at least one graft member comprises two graft members.

79. The system of claim 70 where the graft fixation component of the proximally located anchoring and graft fixation subassembly comprises a ring-like member with an open center and an adjuster passing through said open center and adapted to hold the at least one graft member between the ring-like member and the adjuster.

80. The system of claim 79 where the adjuster comprises a helically grooved member.

81. The system of claim 79 where the adjuster comprises a helically grooved member screw or bolt passing through said ring-like member open center and through the anchor, the screw or bolt having a head opposite the anchor and forming the the graft fixation surface between the ring-like member and the adjuster head.

82. The system of claim 79 where the ring-like member has a size and an outer surface forming the presentation surface.

83. The system of claim 79 where the anchor component comprises a substantially cylindrical member adapted to fit partially into the tunnel and having at least one protuberance to anchor the anchor component to the tunnel.

84. The system of claim 83 where the adjuster is adjustably attachable to the anchor component.

85. The system of claim 83 where the adjuster is adjustably attachable to the anchor component by a nut.

86. The system of claim 70 further comprising a tension measuring component configured to measure tension on the at least one graft member.

87. The system of claim 86 further comprising a tension indicator for displaying tension values on the at least one graft member.

88. An ACL reconstruction system kit combinable with at least one graft member to form a ACL reconstruction system, insertable into a pre-bored tunnel passing from a tibia to a femur, and having an adjustable tension after said insertion comprising:
a.) the distally located anchoring subassembly configured to fix the system to the distal end of the tunnel, and
b.) the proximally located anchoring and graft fixation subassembly having:
i.) engaging surfaces configured to engage an adjustment tool and to adjust tension in the at least one graft member after insertion into the tunnel and anchoring at the distally located anchoring subassembly,
ii.) graft fixation surface, and
iii.) anchoring component configured to anchor the proximally located anchoring and graft fixation subassembly in the proximal end of the pre-bored tunnel.

89. The system of claim 88 in combination with the adjustment tool, the adjustment tool being configured to move the engagement surfaces and to adjust tension on the at least one graft member.

90. The system of claim 88 where the proximally located anchoring and graft fixation subassembly further comprises an adjuster movably attached but fixable to the anchoring component.

91. The system of claim 88 where the proximally located anchoring and graft fixation subassembly comprises a ring-like member with an open center and an adjuster passing through said open center and adapted to hold the at least one graft member between the ring-like member and the adjuster.

92. The system of claim 91 where the adjuster comprises a helically grooved member.

93. The system of claim 91 where the adjuster comprises a helically grooved member screw or bolt passing through said ring-like member open center and through the anchor, the screw or bolt having a head opposite the anchor and forming the the graft fixation surface between the ring-like member and the adjuster head.

94. The system of claim 91 where the ring-like member has a size and an outer surface forming a presentation surface.

95. The system of claim 88 where the anchor component comprises a substantially cylindrical member adapted to fit partially into the tunnel and having at least one protuberance to anchor the anchor component to the tunnel.

96. The system of claim 95 where the adjuster is adjustably attachable to the anchoring component.

97. The system of claim 95 where the adjuster is adjustably attachable to the anchor component by a nut.

98. A method for reconstructing the ACL, comprising the steps of:
a.) constructing a tunnel through a tibia and a femur in said knee,
b.) introducing a system made according to any of claims 1-55 into that tunnel,
c.) anchoring the distally located anchoring subassembly to the distal end of the tunnel,
d.) anchoring the proximally located anchoring and graft fixation subassembly,
e.) adjusting tension of the at least one graft member.

99. The method of claim 98 further comprising the step of flexion and extension of the knee prior to step e.).

100. The method of claim 98 further comprising the step of choosing a ring size adapted to provide a presentation surface to the bone tunnel.
